(19) United States
(12) Patent Application Publication
(10) Pub. No.: US 2006/0105051 A1
(43) Pub. Date: May 18, 2006

(54) Manufacture of particles for pulmonary drug delivery by carbon dioxide assisted nebulization

(76) Inventors: Blair Jackson, South Grafton, MA (US); David Bennett, Stur Bridge, MA (US); Donghao Chen, Lexington, MA (US); Michael M. Lipp, Framingham, MA (US); Purav Dave, San Mateo, CA (US)

Correspondence Address: ELMORE PATENT LAW GROUP, PC, 209 MAIN STREET, N. CHELMSFORD, MA, 01863, US

(21) Appl. No.: 11/260,884

(22) Filed: Oct. 27, 2005

Related U.S. Application Data
(60) Provisional application No. 60/629,122, filed on Nov. 18, 2004.

(51) Int. Cl. A61K 9/14; B29B 9/00

(52) U.S. Cl. 424/489; 264/5

(57) ABSTRACT

Methods of making porous particles by using carbon dioxide assisted nebulization (CAN) technology in combination with spray drying technologies are disclosed. As the mixture of carbon dioxide (CO₂) and the solvent with drug matrix is expanded through the nebulizer to atmospheric conditions, the resulting aerosol contains fine micro-bubbles and/or micro-droplets that contains dissolved CO₂ which is co-currently fed into a spray drying chamber.

### RELATED APPLICATION

This application claims the benefit of U.S. Provisional Application No. 60/629,122, filed on Nov. 18, 2004. The entire teaching of the above application is incorporated herein by reference.

### BACKGROUND OF THE INVENTION

Supercritical fluids have been extensively investigated for use in many common extraction processes. Many supercritical fluid techniques have been used to create small particles for pulmonary delivery, they include rapid expansion of supercritical solution (RESS), supercritical anti-solvent precipitation (SASP), and carbon dioxide assisted-bubble drying (CAN-BD). RESS and CAN-BD utilize the pressure drop across a small orifice to create very small droplets of solution. The droplets dry and can form dry particles.

Supercritical Fluid-Assisted Nebulization and Bubble Drying has been disclosed in U.S. Pat. No. 6,630,121 issued to Sievers, et al. (the '121 patent) In that process, Sievers et al. formed dry particles by forming a composition comprising one or more substances and a supercritical or near critical fluid; reducing the pressure on the compositions thereby forming droplets and passing the droplets through a flow of drying gas which is not the same substances as the supercritical or near critical fluid, said drying gas being heated from above ambient temperature up to 100° C., although the highest temperature Seivers, et al. used was 32° C. (Col. 15, line 9-10). The entire teachings of U.S. Pat. No. 6,630,121 are incorporated herein by reference. However, these particles are not porous particles having desirable properties. Further, the methods of preparing the prior art particles employs a bubble dryer (BD) which operates at temperatures well below inlet temperatures for spray drying apparatus.

There is a need for improved processes for the manufacture of porous particles, in particular in a spray drying process, which (a) increase process-throughput, (b) reduce toxic/organic solvents, (c) allow the combination of compounds completely insoluble in common solvents and/or (d) reducing exposure time in high process temperatures.

There is a need for an improved process for the manufacture which protects fragile agents of spray dried particles, such as bioactive agents, including but not limited to polypeptides, proteins, and peptides.

There is also a need for an improved process for the manufacture of porous particles, in particular, a population of particles with fine particle fractions suitable for their aerodynamic qualities, for example a fine particle fraction of less than 5.6 microns of at least 50%.

### SUMMARY OF THE INVENTION

The present invention provides a method for manufacturing particles comprising feeding a solution into a spray dryer system employing carbon dioxide assisted nebulization wherein the inlet temperature of the spray drying chamber is greater than 100° C. and produces a population of particles having a fine particle fraction less than 5.6 microns of at least 50% (by weight). In one embodiment, the invention is a method wherein population of particles having a fine particle fraction of less than 5.6 microns of at least 50% (by weight) is selected from the group consisting of at least about 53%, 56%, 58%, 60%, 62%, 63%, 66%, 67%, 69%, 73%, 75%, 76%, and 79%. As used throughout this specification, unless noted otherwise, when numbers, value and ranges are listed the term “about” should be inferred even if not specifically written. For example, 60% should be read to include “about 60%”. In another embodiment the population of particles has a fine particle fraction of less than 3.4 microns of at least 20% (by weight). For example, the population of particles has a fine particle fraction of less than 3.4 microns of at least 20% is selected from the group consisting of at least about 21%, 24%, 27%, 28%, 30%, 35%, 37%, 39%, 40%, 41%, 42%, 43%, 44%, 45%, 46%, 47%, 50%, 51%, 55%, 59%, 60%, 63%, 66%, 70%, and 75%.

In yet another embodiment, the invention discloses a method for manufacturing particles comprising feeding a solution into a spray dryer system employing carbon dioxide assisted nebulization wherein the inlet temperature of the spray drier is greater than 100° C. ( as used throughout this document, the inlet temperature of the spray drier must be greater than 100° C. and not read as “greater than about 100° C.”, as such, it is an exception to the note above concerning the use of the term “about”) and produces a population of particles having a fine particle fraction of less than 5.6 microns of at least about 50% to about 80% and fine particle fraction of less than 3.4 microns of at least about 35% to about 75% (by weight). In one embodiment, the population of particles has a fine particle fraction of less than 5.6 microns of at least 53% and fine particle fraction of less than 3.4 microns of at least 35% (by weight). The present invention also provides for a solution comprising a solid compound in a solvent having a concentration from about 3 g/L to about 10 g/L solid compound to solvent. For example, in alternative embodiments the concentration of solid compound in the solution is selected from the group consisting of from about 4 g/L, 5 g/L, 6 g/L, 7 g/L, 8 g/L, 9 g/L and 10 g/L of solid compound to solvent.

The present invention also provides for the feeding of the solution at a solution feed rate in the range from about 25 to about 50 ml/min. In certain embodiments, the solution feed rate is selected from the group consisting of about 25 ml/min, about 30 ml/min, about 35 ml/min, about 40 ml/min, about 45 ml/min and about 50 ml/min.

The method of the invention also employs carbon dioxide assisted nebulization which has a carbon dioxide pressure (psi) in the range from about 1500 to about 2800. In alternative embodiments, the carbon dioxide pressure is selected from the group consisting of about 1500, about 1600, about 1700, about 1800, about 1900, about 2000, about 2100, about 2200, about 2300, about 2400, about 2500, about 2600, about 2700 and about 2800. In one embodiment, the carbon dioxide assisted nebulization employs a tee having an orifice wherein the orifice diameter (μm) is in the range from about 100 μm to about 275 μm, for example, about 100 μm, about 125 μm, about 150 μm, about 175 μm, about 200 μm, about 225 μm and about 250 μm.

The method of the invention provides for an inlet temperature in the range greater than 100° C. to about 140° C. The upper and lower limits of the range can be independently selected and may include temperatures greater than 100° C., about 105° C., about 110° C., about 115° C., about 120° C., about 125° C., about 130° C., about 135° C., and about 140° C. The invention provides for an outlet temperature in the range from about 55° C. to about 75° C. The upper and lower limits to the range can be independently selected. The outlet temperature may include temperatures of about 55° C., about 56° C., about 57° C., about 58° C., about 59° C., about 60° C., about 61° C., about 62° C., and 63° C., about 64° C., about 65° C., about 66° C., about 67° C., about 68° C., about 69° C., about 70° C., about 71° C., about 72° C., about 73° C., about 74° C., and about 75° C.

The present invention further comprises a drying gas which is fed at a drying gas feed rate in the range of about 90 to about 120 kg/hour. The upper and lower limits of the range may be independently selected. For example, the drying gas feed rate includes about 90, about 95, about 100, about 101, about 102, about 103, about 104, about 105, about 106, about 107, about 108, about 109, about 110, about 111, about 112, about 113, about 114, about 115, about 116, about 117, about 118, about 119, or about 120 kg/hour. The invention provides for a drying gas selected from the group consisting of nitrogen, air, carbon dioxide, and mixtures thereof.

### DETAILED DESCRIPTION OF THE INVENTION

Applicants have filed numerous patent applications drawn to various innovations in the spray drying art as it relates to improvements in the production of dry particles. See for example, U.S. Publication number 20030180283 published Sep. 25, 2003 entitled “Method and Apparatus for Producing Dry Particles” which is related to PCT application with the same title PCT/US03/08398 (published as WO 03/080028), entitled “Method and Apparatus for Producing Dry Particles”; U. S. Publication number 20030017113 published Jan. 23, 2003 entitled “Control of process humidity to produce porous particles” and U.S. Publication number 2003222364 with the same title published Dec. 4, 2003. For example, in the above mentioned U.S. Publication 20030017113, Applicants found that particles can be formed which possess targeted aerodynamic properties by controlling the moisture content of a drying gas and contacting the liquid droplets which are formed with the drying gas, thereby drying the liquid droplets to form spray dried particles. The entire teachings of all referenced patent applications, patent publications, journals and any other references throughout this entire application are incorporated herein by reference.

In the method of the invention, one T tube is employed. However, there are circumstances when multiple T tubes (also referred to as T fittings or a tee) are employed. In the context of spray drying, unlike the prior art, combinations of factors of such as, but not limited to, the delicacy of the active agents, the relatively high operating temperatures, the controlled humidity conditions and the need for high throughput must be balanced to arrive at the optimal configuration. In multiple T tube configurations, at least about two to at least about 100 T tubes are employed, for example, two, three, four, five, six, seven, eight, nine or ten T tubes are employed. In the invention, at least about 25, at least about 50 up to at least about 100 T tubes may be employed. The upper and lower limits of the range may be independently selected. The arrangement of a multiple T tube configuration is designed for optimal placement in the spray drying system and can be linear, circular, patterned (such as, for example, a square-, rectangular-, hexagonal- or diamond-shaped), irregularly located. In designing the optimal configuration, the capacity of spray dryer is a crucial consideration. One the one hand, prior art spray drying operations cannot have more than one atomizer because the atomizers, such as rotary atomizers or other presently employed nozzles, spray onto each other. That is, they spin or rotate causing their widely dispersed sprays comes out at droplets which can contact droplets from other nozzles. Before the present multiple T tube configuration of the invention, if multiple atomizers were employed, droplets would come in contact with one another changing the size of the droplets and otherwise comprising the homogeneity of the droplet size. As such, the current atomizers, for example rotary atomizers, do not take advantage of the capacity of the drying chamber. The use of multiple T tubes in the method of the invention increases the drying capacity of the spray dryer thereby increasing output, decreasing costs, decreasing waste from the system and other such advantages. Further using multiple T tubes allows (1) the use of more concentrated solutions, (2) making the runs quicker, (3) the use of higher temperature and (4) speeding up the drying process overall. The balancing of all the necessary conditions, however, is not trivial.

Applicants have improved the dry powder technology, especially methods of manufacture using spray drying apparatus. For example, a modified CAN atomization process was conducted by pumping liquid CO₂ into a restrictor from 1200 PSI to the desired set point using a high pressure pump (Thar Technologies, Pittsburg, Pa.). The solids stream was fed to the restrictor using an HPLC pump (ChromTech, Apple Valley, Minn.). The two streams met in a low dead volume T fitting that was housed on top of a spray dryer, for example a Niro size 1 spray dryer. Although a Size 1 spray dryer was employed, those skilled in the art will appreciate that the methods of the invention can be adapted to other size and brands of spray dryers. The T is maintained at a temperature calculated to maintain the temperature of the stream(s) entering the T. The conditions used to dry the droplets are discussed further herein. In this application, the Applicants discloses alternative methods of producing particles, especially populations of particles, having desired fine particle fractions are suitable for administration by inhalation. In certain embodiments the particles are porous particles and have a volume median geometric diameter (VMGD) from about 5 to about 30 microns and tap density less than about 0.4 g/cm³, less than about 0.3 g/cm³, less than about 0.2 g/cm³, or less than about 0.1 g/cm³. Such particles possess suitable aerosol performance properties that increase deep lung deposition and enhance the bioavailability of therapeutic agents. Strategically combining manufacturing parameters, including adapting the CAN atomization process, permits the manufacture of selected populations of particles. Still further, this disclosure generally relates to a method of making porous particles for pulmonary delivery of therapeutic and diagnostic agents; more particularly, it is based on the unexpected discovery of making porous particles by using carbon dioxide assisted nebulization (CAN) technology. The invention exploits the varying solubilities of solvents coupled with the expansion of the resulting emulsion of drug through a nebulizer. As the mixture of carbon dioxide (CO₂) and the solvent with drug matrix is expanded through the nebulizer to atmospheric conditions, the resulting aerosol contains fine micro-bubbles and/or micro-droplets that contains dissolved CO₂. While not wishing to be bound to a single theory, the dissolved CO₂ is thought to undergo a subsequent expansion, rupturing the droplets into extremely fine particles and pulling the solvent off the particles leaving no toxic residue on the dried particles and eliminating concerns of waste solvent disposal. The ability to adjust temperature and pressure of the supercritical solution can lead to increases in solubility of about 3 to about 10 orders of magnitude compared to common solvents and, if desired, can aid very poorly soluble compounds. The aerosol containing fine micro-bubbles and/or micro-droplets that contains dissolved CO₂ is fed into a drying chamber as described below. The T is heated to keep all the fluids flowing through at a constant temperature. Preferably, the apparatus is jacketed where appropriate to maintain the desired temperature. The jacketing may be permanent or removable as desired to regulate the temperature.

A drug, also referred to as an active agent or agent, includes but is not limited to a small molecule, protein, peptide, macromolecule, nuclei acid, or any other compound useful in the treatment, prophylaxis or diagnosis of disease. Examples of suitable drugs can be any of the many compounds being tested by drug delivery companies, such as but certainly not limited to, insulin, hGH, FSH, PTH, epinephrine, budesonide, fluticasone, trospium, ipatropium bromide and other such agent. Examples of other suitable agents and excipients are listed in, for example, U.S. Pat. No. 6,749,835 to Lipp et al. issued Jun. 15, 2004, U.S. Pat. No. 6,586,008 to Batycky et al. issued Jul. 1, 2003 and U.S. Pat. No. 6,095,134 to Sievers et al. issued on Aug. 1, 2000.

In the invention, the CAN process is employed. For example, a co-solvent of ethanol and water with various ratios were used, and a formulation solution was mixed in a low dead volume tee with supercritical CO₂ of pressure from about 1500 psi to about 2800 psi to form an emulsion. The emulsion was then rapidly expanded and atomized through a flow restrictor into a drying chamber, for example, a spray dryer chamber which was operated at atmospheric pressure; the resulting micro-droplets and/or micro-bubbles then were dried by warmed gas, for example warmed nitrogen gas, and particles were collected in the collection apparatus, for example, in a bagfilter, cyclone or other such collecting apparatus.

When certain collecting apparatus is used, the collecting apparatus is pulsed and the powder falls into an awaiting collection vessel. Using the teachings of the present invention, one skilled in the art will appreciate spray dryers such as but not limited to Niro spray drying systems can be employed. An example of a suitable system which may be employed is disclosed in U.S. Ser. No. 60/545,048 filed Feb. 17, 2004 entitled “Method and Apparatus for Producing Dry Particles” (Atty. Docket No. 2685.3003 US).

The combination of CAN technology and spray drying, especially if closed system is employed offers several advantages over conventional spray drying in that the throughput of the manufacturing process can be increased greatly while decreasing the amount of volatile solvents released in the manufacturing process. Even in an open system, the efficiency of manufacture is increased and the amount of excipients, agents and/or solvents can be decreased. In the method of the invention, the CAN technology is combined with any number of spray dryers, including but not limited to, conventional two-stage spray dryer, compact spray dryer, fluidized spray dryers, multi-stage dryers and the like. The method of the invention can be practiced in small, medium or large scale production of particles. Those of skill in the art will appreciate the features of commercially available spray driers suitable for the practice of the invention. For example, Niro A/S of Denmark produces many models of spray drier which may be adapted for use in the methods of the invention. Also, disclosed is the novel combination of the CAN technology with the proprietary spray-drying process described in co-pending Provisional Application No: 60/545,048 entitled “Method and Apparatus for Producing Dry Particles” (Atty. Docket 2685.3003 US).

The CAN atomization process can be conducted by pumping liquid into a tee. For example, a low volume tee is used instead of a standard atomization nozzle, including but not limited to a rotary atomizer or a two-fluid nozzle.

In the invention, solutions, preferably insoluble compound-containing solutions, are employed. For example, suitable solutions include solutions having volatile salts as components which are co-currently fed through a combination system. The solutions of the invention comprise various ratios from, for example, 100% water, 30/70 (v/v) ethanol/water, 40/60 ethanol/water, 50/50 ethanol/water or 60/40 ethanol/water. Other solvents include but are not limited to acetone, methylene chloride, ethanol, and other organic solvents in combination with water in varying concentrations or neat.

The particles produced by the method of the invention may be porous particles. Examples of porous particles produced include particles having any of the following combinations of desirable features which may include at least two of the following features a targeted VMGD, a targeted FPF, a targeted tap density or any combination of such features, including but not limited to:

(a) VMGD of greater than 5 microns and at least 54% FPF<5.6 μm;

(b) VMGD of at least 6.5 to 15.5 microns at 1.0 bar (μm), at least 20% FPF<3.3 μm, also having a tap density of less than 0.4, less than 0.3, less than 0.2 g/cm³;

(c) VMGD pf at least 15.5 microns at 1.0 bar (elm), a FPF<3.3 μm of at least 11%, also having a tap density of less than 0.4 g/cm³, less than 0.3 g/cm³, less than 0.2 g/cm³ or less than 0.1 g/cm³;

(d) VMGD from about 5.7 μm to about 15.5 μm and a FPF<5.6 of at least about 53%.

In other embodiments, methods of the invention are employed to produce a population of particles having a fine particle fraction less than 5.6 microns of at least 50% (by weight).

This invention includes a method of manufacturing particles, in particular porous particles, comprising feeding a solution into a drying system employing carbon dioxide assisted nebulization (CAN) technology wherein the inlet temperature of the spray drier is greater than 100° C. and producing a population of particles having a fine particle fraction of less than 5.6 microns of at least 50% (by weight), including at least 50%, 51%, 52%, 53%, 54%, 55%, 59%, 60%, 63%, 66%, 70%, 75%, 76%, 78%, 80%, 85%, 90%, 95%, 96%, 97% or more.

The method of the invention produces a population of particles having a fine particle fraction of less than 3.4 microns of at least 20% (by weight), including at least about 21%, 22%, 23%, 24%, 25%, 26%, 27%, 28%, 29%, 30%, 35%, 37%, 39%, 40%, 41%, 42%, 43%, 44%, 45%, 46%, 47%, 50%, 51%, 55%, 59%, 60%, 63%, 66%, 70%, 75%, 76%, 78%, 80%, 85%, 90%, 95% or more.

The method of the invention produces a population of particles having a fine particle fraction of less than 5.6 microns of at least about 50% to about 80% and fine particle fraction of less than 3.4 microns of at least about 35% to about 75% (by weight). In one embodiment, the method produces a population of particles having a fine particle fraction of less than 5.6 microns of at least 53% and fine particle fraction of less than 3.4 microns of at least 35% (by weight).

In another embodiment, a solution used in the preparation of the particles of the invention is a “poorly soluble component-containing solution” comprises a solution having a solid compound, in particular an agent which is poorly soluble, including insoluble, in its solvent for example, water, ethanol/water solution or in a selected liquid medium. The concentration of the solid compound is from about 3 g/L to about 10 g/L, in particular from about 4 g/L, 5 g/L, 6 g/L, 7 g/L, 8 g/L, 9 g/L or 10 g/L of solid compound to solvent. The upper and lower limits of the range can be selected independently. Suitable agents are discussed above.

The method of the instant invention operates most effectively when the process conditions are selected from combinations of following conditions:

Liquid flow (ml/min) of the solution is in the range from about 10 to about 50 ml/min, in particular, about 10 ml/min, about 20 m/min, about 25 ml/min, about 30 ml/min, about 35 ml/min, about 40 ml/min, about 41 ml/min, about 42 ml/min, about 43 ml/min, about 44 ml/min, about 45 ml/min, about 46 ml/min, about 47 ml/min, about 48 ml/min, about 49 min/min or about 50 ml/min. The upper and lower limits of the range can be selected independently. This solution can be fed into the spray dryer system at back pressures sufficient to achieve these liquid flow rates, for example greater than about 1000 PSI, greater than 1100 PSI, greater than 1200 PSI, greater than 1300 PSI, greater than 1400 PSI and greater than 1500 PSI. The upper and lower limits of the range can be selected independently. These flow rates and back pressures can be achieved with pumps and tubing connections known to those skilled in the art. For instance, known HPLC pumps and tubing can be used.

Carbon dioxide pressure (psi) is at least about 1500, for example, 1500, 1600, 1700, 1800, 1900, 2000, 2100, 2200, 2300, 2400, 2500, 2600, 2700 or 2800. Carbon dioxide pressure (psi) is less than about 2900. The upper and lower limits of the range can be selected independently.

Orifice Diameter (μm) is in the range from about 100 μm to about 275 μm, for example, 100 μm, 125 μm, 150 μm, 175 μm, 200 μm, 225 μm or 250 μm. The upper and lower limits of the range can be selected independently.

Suitable inlet temperatures are in the range of greater than 100° C. to about 140° C. depending upon the formulation and the other conditions. Inlet temperatures of greater than about 101° C., 105° C., 110° C., 115° C., 120° C., 125° C., 130° C., 135° C., or 140° C., are suitable. The upper and lower limits of the range can be selected independently.

Suitable outlet temperatures are in the range from about 55° C. to about 75° C. including but not limited to about 55° C., 56° C., 57° C., 58° C., 59° C., 60° C., 63° C., 64° C, 65° C., 66° C., 67° C., 68° C., 69° C., 70° C., 71° C., 72° C., 73° C., or 75° C. The upper and lower limits of the range can be selected independently.

The drying gas is fed in the range of about 90 to about 120 kg/hour, for example, about 90, about 95, about 100, about 101, about 102, about 103, about 104, about 105, about 106, about 107, about 108, about 109, about 110, about 111, about 112, about 113, about 114, about 115, about 116, about 117, about 18, about 119, or about 120 kg/hour. The upper and lower limits of the range can be selected independently. The drying gas in the method of the invention is selected from the group consisting of nitrogen, air, carbon dioxide or other suitable spray drying gases know to those skilled in the art. Mixtures of gases may also be employed.

Formulations have been tested to confirm that the porous particles can be made by the CAN technology when used in combination with a spray dryer. The Applicants has optimized the conditions, apparatus and formulations in such a way as to suggest a pattern which will revolutionize the manner in which drug delivery companies produce dry powder particles. Various operating parameters, including size of the restrictor, pressure of supercritical CO₂ and liquid flow rate have been tested in order to make porous particles. VMGD and FPF have been characterized.

Any techniques for characterizing particles and populations of particles are known to those skilled in the art. The below descriptions of some suitable techniques are intended to be instructive and non-limiting.

Measuring the Fine Particle Fraction

The two stage Anderson Cascade Impactor (ACI-2) determines fine particle fraction of a powder, for example, by using Gravimetric Analysis.

An ACI-2 was assembled using stages 0, 2, and F from a standard Anderson Cascade Impactor. A 90 mm, 0.5 μm pore size, glass fiber filter (Pall Gelman Sciences) was placed on the plate above stage F as well as on stage F. The apparatus was attached to a flowmeter, needle valve, and vacuum pump. An inhaler with a blank capsule was placed in the induction port and the vacuum pump was turned on. The needle valve was adjusted until the flowmeter read 60 LPM. This flow rate results in all particles of <3.4 μm landing on stage F and >3.4 μm and <5.6 μm landing on stage 2.

A size 0 Hydroxypropylmethylcellulose (HPMC) capsule was filled with 10+1 mg of powder. The capsule was placed in the inhaler and actuated. The inhaler was then placed into the induction port and the vacuum pump was operated for 2 seconds. The filters were weighed and the mass of powder on each stage determined. A formula for calculating the FPF<3.4 μm is:
FPF<3.4μm=(filter weight_{final, stage F}-filter weight_{initial, stage F})/capsule fill weight
A formula for calculating the FPF <5.6 μm is:
FPF<5.6 μm =FPF<3.4 μm+(filter weight_{final, stage 2}-filter weight_{initial, stage 2})/capsule fill weight.
Emitted Dose

The emitted dose test determines the percentage of powder that will emit from an inhaler for example by using gravimetric analysis. An inhaler, for example that described in U.S. Pat. No. 6,732,732 to Edwards et al. on May 11, 2004 may be used however other suitable inhalers are known to those skilled in the art.

A filter was placed on the base of the emitted dose apparatus. The port was placed onto the base and the end was connected to a flowmeter, needle value, and vacuum pump. An inhaler with an empty capsule was placed in the port. The vacuum pump was turned on and the needle valve adjusted until the flow was 60 LPM.

A filled capsule (10±1 mg) was placed into an inhaler. The inhaler was actuated and placed into the port. The vacuum pump was turned on for 2 seconds. The filter was weighted and the mass of powder determined. A formula for finding emitted dose is seen below.
ED=(filter weight_{final}-filter weightᵢₙᵢₜᵢₐₗ)/capsule fill weight
Volume Median Geometric Size of the Emitted Dose (IHA): RODOS The IHA test determines the volume median geometric diameter (VMGD) of an emitted powder, using light diffraction, for example, obtaining the geometric particle size distribution of the emitted dose using the Helos Laser Diffractometer and Inhaler Attachment.

Powder was placed into a HPMC size 0 capsule and the capsule was placed into an inhaler. The inhaler was actuated and placed into a housing specially designed for this process. The housing allows compressed air to be pushed through the inhaler, simulating a breath. A compressed air line was connected to the housing and passed through a valve and flow meter. The system was set to run at 60 LPM for 2 seconds.

When the valve is opened, the powder emits from the capsule and the VMGD is measured by laser diffraction.

Volume Median Geometric Diameter of a Bulk Powder: HELOS/RODOS

The HELOS/RODOS test uses laser diffraction to measure the volume median geometric diameter of a bulk powder. In addition, running the machine at different shear rates (pressures), the dispersibility of a powder can be determined.

A pressure and corresponding vacuum ar entered into the RODOS equipment. The test begins by setting the RODOS to 0.5 bar dispersion pressure. A reference of the laser diffraction was taken, and then a small amount of powder was put into the flow stream. The powder passed through the laser's beam and a diffraction pattern was obtained. This diffraction pattern correlates to a geometric size. This test can also be run at 1.0, 2.0, and 4.0 bar to determine how shear affects the geometric size of the powder.

Tap Density

A VanKel Tap Density Tester, model 5-1442-0398, was used for the tap density tests. A small centrifuge vial was tared and then filled with powder and reweighed. The vial was placed into the machine and tapped 1000 times. The vial was removed, and a second vial of the same size was tared. Water was added to the second vial until it reached the same level as the powder. The water and vial were weighed. Assuming unit density for the water, the formula for calculation tap density is:
Tap Density=Mass_{powder}/Mass_{water}
Scanning Electron Microscope (SEM)

The scanning electron microscope (SEM) was used to visually inspect the particles and observed morphology.

The powders were lightly tapped onto a tape strip on a SEM stage. Compressed air was blown over the surface to obtain a monolayer of particles on the surface.

The powders were then gold coated using a Polaron model SC7620 sputter coater. A Personal SEM from ASPEX, LLC was used for the study. The powders were observed at low (33×) through high (5000×) magnification and images were taken of various particles.

Dynamic Scanning Calorimetry (DSC)

Dynamic scanning calorimetry (DSC) is a technique used to observe temperature events in a sample.

DSC was performed using a TA Instruments Series 2920 Differential Scanning Calorimeter. Indium metal was used as a calibration standard. The sample was sealed in hermetic aluminum DSC pans. The sample was equilibrated to 10° C. and held isothermally for 1 minute. The powders were then heated to 10° C./min to a final temperature of 250° C.

The transition temperature was determined to be the temperature at the signal maximum for a change in heat flow. The onset temperature was determined from the intercept of the baseline with the steepest tangent to the left side of the transition peak.

Thermal Gravimetric Analysis (TGA)

Thermal gravimetric analysis (TGA) is a test to measure residual water content of a powder.

TGA was performed using a TA Instrument Series 2050 Thermogravametric Analyzer. Samples for TGA moisture analysis were heated from ambient temperature to 250° C. at 20° C./min. The moisture content was determined by monitoring the percent of weight loss of the sample between ambient temperature and 150° C.

Residual Ethanol

The residual ethanol was tested using a capillary gas chromatograph. 20 mg of sample was placed in a vial and 150 μL of dioxane was added to dissolve the sample. The sample was loaded into the chromatograph and analyzed.

The below described compounds were used in the application of the CAN technology to the art of spray drying techniques for the production of powders. For purposes of this application, this novel combination is sometimes referred to herein as CAN-SD or the CAN-SD process. Leucine is representative of a small molecule excipient used in a formulation. Albumin (BSA) is both a model of a protein therapeutic and a large macromolecule excipient. Powders were produced utilizing both the CAN-SD process and a conventional two-fluid atomizer and compared for the following properties: VMGD, FPF and morphology. As used herein in the Example section, unless otherwise noted, the terms “conventional”, “2-fluid atomizer process” and “conventional two-fluid atomizer” are used interchangeably. It is understood however, using the teachings herein, that other conventional spray drying techniques could be employed in the practice of the invention by those of skill in the art.

Other powders such as epinephrine were prepared using the methods of the invention. For example, particles having a composition in the range of about 70 to about 75 weight percent leucine, from about 15 to about 20 weight percent epinephrine bitartrate, and from about 10 to about 15 weight percent sodium tartrate. In one embodiment, the particles have a composition of 72 weight percent leucine, 16 weight percent epinephrine bitartrate, and 12 weight percent sodium tartrate. In another embodiment, the formulation was 73.34 weight percent leucine, 16.1 weight percent epinephrine bitartrate, and 10.56 weight percent sodium tartrate.

General Experimental Design

The experimental design was generally as described below. Certain modifications were made as also described in more detail herein. Experimental designs were employed for the production of the leucine and BSA powders via the CAN process to determine which of the processing conditions significantly influence properties of the powders produced using the CAN process. One skilled in the art using the conditions set forth will appreciate that the methods of the invention can be employed to prepare a variety of powders with at least the same or better particle characteristics using less starting material and releasing fewer environmentally sensitive by-products. Applicants disclose other powders which were produced as described further herein.

TABLE 1
DRYING CONDITIONS FOR LEUCINE/BSA CAN RUNS.

| | Leucine | BSA |
|---|---|---|
| Inlet Temp [C.] | 105-125 | 100-120 |
| Outlet Temp [C.] | 60-65 | 60-65 |
| Liquid Flow [mL/min] | 25-50 | 25-50 |
| Drying gas [kg/hr] | 100 | 100 |
| CO₂ Pressure [psi] | 1500-2500 | 1500-2700 |
| Chamber Pressure [in H₂0] | −2 | −2 |
| Nozzle orifice [μm] | 125, 175, 250 | 125, 175, 250 |

Two-Fluid Nozzle Atomization - Leucine and BSA Powders

The solutions were spray dried using a Niro Size 1 spray dryer. The formulations were tested using the 2-fluid nozzle and the powder was collected on the baghouse. The conditions were used to make the powders are displayed in Table 2.

TABLE 2
CONDITIONS USED WITH 2-FLUID
NOZZLE ATOMIZATION.

| | Leucine | BSA |
|---|---|---|
| Inlet Temp [C.] | 150 | 160 |
| Outlet Temp [C.] | 60 | 69 |
| Liquid Flow [mL/min] | 60 | 60 |
| Drying gas [kg/hr] | 100 | 100 |
| Atomization gas [g/min] | 30 | 30 |
| Chamber Pressure [in H₂O] | −2 | −2 |
| Nozzle Configuration | 2850/67147 | 2850/67148 |

Further examples of conventional processes are found in U.S. Ser. No. 10/607,571 (Atty. Docket No. 2685-2046 US3) and PCT/US03/20166 (Atty. Docket 2658-2046 WO) the entire teachings of which are incorporated herein by reference.

Leucine Only

A formulation comprising 100% leucine was used for this study. The formulation was prepared utilizing a solids concentration of 8 g/L of leucine in a solvent comprised of 50/50 v/v EtOH/H₂O. The leucine was added to the aqueous phase and heated until it was completely dissolved. The ethanol was also heated to 60° C. and then slowly added to the aqueous solution.

TABLE 3
CAN-BD

| | Leucine |
|---|---|
| Inlet Temp [C.] | 105-125 |
| Outlet Temp [C.] | 60-65 |
| Liquid Flow [mL/min] | 25-50 |
| Drying gas [kg/hr] | 100 |
| CO₂ Pressure [psi] | 1500-2500 |
| Chamber Pressure [in H₂0] | −2 |
| Nozzle orifice [μm] | 125, 175, 250 |

TABLE 4
CONDITIONS USED WITH 2-FLUID
NOZZLE ATOMIZATION

| | Leucine |
|---|---|
| Inlet Temp [C.] | 150 |
| Outlet Temp [C.] | 60 |
| Liquid Flow [mL/min] | 60 |
| Drying gas [kg/hr] | 100 |
| Atomization gas [g/min] | 30 |
| Chamber Pressure [in H₂O] | −2 |

TABLE 5
LEUCINE POWDER PROPERTIES AT
VARIOUS PROCESS CONDITIONS

| | Liquid | CO₂ | Orifice | | |
|---|---|---|---|---|---|
| | flow | pressure | diameter | VMGD | |
| Run # | (ml/min) | (psi) | (μm) | (μm) | FPF < 5.6 μm |
| 1 | 25 | 2200-2500 | 125 | 8.69 | 56 |
| 3 | 25 | 2200-2500 | 175 | 5.7 | 66 |
| 6 | 25 | 2200-2500 | 250 | 8.88 | 59 |
| 2 | 25 | 1500-1800 | 175 | 6.88 | 63 |
| 5 | 25 | 1500-1800 | 250 | 10.22 | 53 |
| 4 | 35 | 2200-2500 | 175 | 6.5 | 63 |
| 7 | 35 | 2200-2500 | 250 | 7.76 | 63 |
| 8 | 35 | 2200-2500 | 250 | 8.68 | 62 |
| 9 | 50 | 2200-2500 | 250 | 8.11 | 66 |

Table 5 shows the VMGD and FPF results of Leucine formulation. It is clear that at all operating conditions, the VMGD of every powder collected is larger than 5 microns and all have FPF greater than 50%.

The comparison of the FPF values of the powders made by the CAN-SD process versus conventional spray drying process is shown in Table 6. It can be found that powders made using CAN technology have better properties in either or both FPF and VMGD. This clearly demonstrates the improvement of the present combination technology when compared to conventional spray drying.

TABLE 6
COMPARISON OF LEUCINE POWDER MADE VIA
THE CAN AND CONVENTIONAL PROCESS

| | VMGD | FPF < 5.6 μm | FPF < 3.4 μm |
|---|---|---|---|
| Powder | (μm) | (%) | (%) |
| Conventional process | 8.31 | 58 | 39.6 |
| CAN Formulation 1 | 8.69 | 56 | 37 |
| CAN Formulation 2 | 6.88 | 63 | 43 |
| CAN Formulation 3 | 5.7 | 66 | 52 |
| CAN Formulation 4 | 6.5 | 63 | 45.9 |
| CAN Formulation 5 | 10.22 | 53 | 35.4 |
| CAN Formulation 6 | 8.88 | 59 | 43.9 |
| CAN Formulation 7 | 7.76 | 63 | 43.2 |
| CAN Formulation 8 | 8.68 | 62 | 42.3 |
| CAN Formulation 9 | 8.11 | 66 | 44.3 |

Leucine powders (100%) were successfully produced utilizing the CAN process. Increasing the orifice size utilized in the CAN process resulted in an increase in the final particle size.

Bovine Serum Albumin

Another formulation studied was 100% bovine serum album (BSA) with ammonium bicarbonate used as a volatile salt (not present in the final powder). The formulation was prepared utilizing a solids concentration of 4 g/L of BSA with 10 g/L of ammonium bicarbonate. The solvent system was comprised of 40/60 v/v EtOH/H₂O. The aqueous and ethanolic phases were both heated to 60° C.

TABLE 7
DRYING CONDITIONS FOR BSA CAN RUNS.

| | BSA |
|---|---|
| Inlet Temp [C.] | Greater than 100 to 200 |
| Outlet Temp [C.] | 60-65 |
| Liquid Flow [mL/min] | 25-50 |
| Drying gas [kg/hr] | 100 |
| CO₂ Pressure [psi] | 1500-2700 |
| Chamber Pressure [in H₂0] | −2 |
| Nozzle orifice [μm] | 125, 175, 250 |

TABLE 8
CONDITIONS USED WITH 2-FLUID
NOZZLE ATOMIZATION

| | BSA |
|---|---|
| Inlet Temp [C.] | 160 |
| Outlet Temp [C.] | 69 |
| Liquid Flow [mL/min] | 60 |
| Drying gas [kg/hr] | 100 |
| Atomization gas [g/min] | 30 |
| Chamber Pressure [in H₂O] | −2 |
| Nozzle Configuration | 2850/67148 |

Table 9 and Table 10 show the results of BSA formulation in which the CAN technology presents its advantage over the conventional spray drying process in aerosol properties.

TABLE 9
BSA POWDER AEROSOL PROPERTIES
MADE VIA THE CAN PROCESS

| | Liquid | CO₂ | Orifice | | FPF < |
|---|---|---|---|---|---|
| | flow | pressure | diameter | VMGD | 5.6 μm |
| Formulation | (mL/min) | (psi) | (μm) | (μm) | (%) |
| CAN | 25 | 1500-1800 | 175 | 9.49 | 69 |
| Formulation 2 | | | | | |
| CAN | 25 | 2200-2500 | 175 | 9.56 | 67 |
| Formulation 3 | | | | | |
| CAN | 35 | 2200-2500 | 175 | 11.6 | 73 |
| Formulation 4 | | | | | |
| CAN | 35 | 1500-1800 | 250 | 5.07 | 79 |
| Formulation 7 | | | | | |

TABLE 10
COMPARISON OF AEROSOL PROPERTIES OF BSA POWDER
MADE VIA THE CAN AND CONVENTIONAL PROCESSES

| | VMGD | FPF < 5.6 μm | FPF < 3.4 μm |
|---|---|---|---|
| Powder | (μm) | (%) | (%) |
| Conventional process | 6.55 | 55.6 | 39.8 |
| CAN Formulation 2 | 9.49 | 69 | 59 |
| CAN Formulation 7 | 5.07 | 79 | 66 |
| CAN Formulation 3 | 9.56 | 67 | 59 |
| CAN Formulation 4 | 11.9 | 73 | 63 |

EPI
Epinephrine Produced by CAN Method of the Invention

A formulation of epinephrine bitartrate was used. The formulation was prepared utilizing the various solids concentrations listed in the table below. The various process conditions are also indicated below.

TABLE 11

| | | | SPRAY DRYING CONDITIONS | | | | |
|---|---|---|---|---|---|---|---|
| | NOZZLE | SOLIDS | | | | CO₂ | DRYING |
| SR. | DIA. | CONC. | T_{IN} | T_{OUT} | LIQ. FEED | PRESSURE | GAS RATE |
| # | (μM) | (G/L) | (° C.) | (° C.) | (ML/MIN) | (BAR) | (KG/HR) |
| 3 | 175 | 2 | 102 | 66.8 | 21.5 | 60 | 92.5 |
| 5 | 175 | 2 | 104 | 65.1 | 20.0 | 70 | 78.0 |

The above-described CAN-SD process was employed in the preparation of particles having the formulation was 73.34 weight percent leucine, 16.1 weight percent epinephrine bitartrate, and 10.56 weight percent sodium tartrate.
Spray Drying Epinephrine Using 2-Fluid Nozzle (Conventional)

This example describes the preparation of particles having the composition of 72 weight percent leucine, 16 weight percent epinephrine bitartrate, and 12 weight percent sodium tartrate. Additionally, the preparation of particles having the composition of having the formulation was 73.34 weight percent leucine, 16.1 weight percent epinephrine bitartrate, and 10.56 weight percent sodium tartrate.

300 mL of an aqueous solution containing 0.9 g epinephrine bitartrate and 4.1 g leucine in water (Sterile Water for Irrigation, USP) was prepared. The pH of the aqueous solution was adjusted to 4.3 by the addition of sodium tartrate. A spray-drying feed solution was prepared by in-line static mixing the aqueous solution with 700 mL of ethanol solution (200 proof, USP), while maintaining both solutions at room temperature. The resulting combined aqueous/organic feed solution was pumped at a controlled rate of 65 mL/min into the top of the spray-drying chamber. Upon entering the spray-drying chamber, the solution was atomized into small droplets of liquid using a 2 fluid atomizer at a rate of 23.5 g/min (CPS,PD), 19.5 g/min (RD). The process gas, heated nitrogen, was also introduced at a controlled rate of 100 kg/hr into the top of the drying chamber of Niro spray dryer (Model PSD-1). As the liquid droplets contacted the heated nitrogen, the liquid evaporated and porous particles were formed. The temperature of the inlet was 107° C. and the outlet temperature was 47° C. The particles exited the drying chamber with the process gas and entered a powder product filter downstream. The product filter separated the porous particles from the process gas stream, including the evaporated solvents. The process gas exited from the top of the collector and was directed to the exhaust system. The porous particles exited from the bottom of the product filter and were recovered in a powder collection vessel as dry powder particles. The resulting particles have a VMGD of 5.3 microns at 1 bar as determined by RODOS and an FPF(<3.3) of 25 to 30% using ACI-3 with wet screens. The dry powder was filled into size 2 hydroxypropylmethyl cellulose (HPMC) capsules and then packaged. Multiple runs were made and similar measurements were found.

While this invention has been particularly shown and described with references to preferred embodiments thereof, it will be understood by those skilled in the art that various changes in form and details may be made therein without departing from the scope of the invention encompassed by the appended claims.

We claim:

1. A method for manufacturing particles comprising feeding a solution into a spray dryer system employing carbon dioxide assisted nebulization wherein the inlet temperature of the spray drying chamber is greater than 100° C. and produces a population of particles having a fine particle fraction less than 5.6 microns of at least 50% (by weight).

2. The method of claim 1, wherein the population of particles has a fine particle fraction of less than 5.6 microns of at least 50% (by weight) is selected from the group consisting of at least about 50%, 51%, 52%, 53%, 54%, 55%, 59%, 60%, 63%, 66%, 70%, 75%, 76%, 78%, 80%, 85%, 90%, 95%, 96%, and 97%.

3. The method of claim 1, wherein the population of particles has a fine particle fraction of less than 3.4 microns of at least 20% (by weight).

4. The method of claim 3, wherein the fine particle fraction of less than 3.4 microns of at least 20% is selected from the group consisting of at least 21%, 22%, 23%, 24%, 25%, 26%, 27%, 28%, 29%, 30%, 35%, 37%, 39%, 40%, 41%, 42%, 43%, 44%, 45%, 46%, 47%, 50%, 51%, 55%, 59%, 60%, 63%, 66%, 70%, and 75%.

5. A method of manufacturing particles comprising feeding a solution into a spray dryer system employing carbon dioxide assisted nebulization wherein the inlet temperature of the spray drier is greater than 100° C. and produces a population of particles having a fine particle fraction of less than 5.6 microns of at least about 50% to about 80%.

6. The method of claim 5 further comprising a fine particle fraction of less than 3.4 microns of at least about 35% to about 75% (by weight).

7. The method of claim 5, wherein the population of particles has a fine particle fraction of less than 5.6 microns of at least about 53% and fine particle fraction of less than 3.4 microns of at least about 35% (by weight).

8. The method of claim 1, wherein the solution comprises a solid compound in a solvent having a concentration from about 3g/L to about 10 g/L solid compound to solvent.

9. The method of claim 1, wherein the solution comprises solid compound in a solvent having a concentration selected from the group consisting of about 4 g/L, about 5 g/L, about 6 g/L, about 7 g/L, about 8 g/L, about 9 g/L and about 10 g/L of solid compound to solvent.

10. The method of claim 1, wherein the feeding of the solution is at a solution feed rate in the range from about 25 to about 50 ml/min.

11. The method of claim 1, wherein the feeding of the solution is at a solution feed rate selected from the group consisting of about 25 ml/min, about 30 ml/min, about 35 ml/min, about 40 ml/min, about 45 ml/min and about 50 ml/min.

12. The method of claim 1, wherein the carbon dioxide assisted nebulization has a carbon dioxide pressure (psi) in the range from about 1500 to about 2800.

13. The method of claim 12, wherein the carbon dioxide pressure is selected from the group consisting of about 1500, about 1600, about 1700, about 1800, about 1900, about 2000, about 2100, about 2200, about 2300, about 2400, about 2500, about 2600, about 2700 and about 2800.

14. The method of claim 1, wherein the carbon dioxide assisted nebulization comprises a tee having an orifice wherein the orifice diameter (μum) is in the range from about 100 μm to about 275 μm.

15. The method of claim 14, wherein the orifice diameter is selected from the group consisting of about 100 μm, about 125 μm, about 150 μm, about 175 μm, about 200 μm, about 225 μm and about 250 μm.

16. The method of claim 1, wherein the inlet temperature in the range greater than 100° C.

17. The method of claim 16, wherein the inlet temperature is selected from the group consisting of greater than about 101° C., about 105° C., about 110° C., about 115° C., about 120° C., about 125° C., about 130° C., about 135° C., and about 140° C.

18. The method of claim 1 further comprising an outlet temperature in the range from about 55° C. to about 75° C.

19. The method of claim 18, wherein the outlet temperature is selected from the group consisting of 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., and about 75° C.

20. The method of claim 1 further comprising a drying gas which is fed at a drying gas feed rate in the range of about 90 to about 120 kg/hour.

21. The method of claim 20, wherein the drying gas feed rate is selected from the group consisting of about 90, about 95, about 100, about 101, about 102, about 103, about 104, about 105, about 106, about 107, about 108, about 109, about 110, about 111, about 112, about 113, about 114, about 115, about 116, about 117, about 118, about 119, or about 120 kg/hour.

22. The method of claim 20, wherein the drying gas selected from the group consisting of nitrogen, air, carbon dioxide and mixtures thereof.

23. The method of claim 1, wherein the carbon dioxide assisted nebulization comprises one or more tees each having an orifice wherein the orifice diameters (em) are in the range from about 100 μm to about 275 μm.

24. The method of claim 22, wherein the orifice diameters (μm) are selected from the group consisting of about 100 μm, about 125 μm, about 150 μm, about 175 μm, about 200 μm, about 225 μm and about 250 μm.

25. A composition prepared using the method of claim 1.
